(19) United States
(12) Patent Application Publication
(10) Pub. No.: US 2006/0240037 A1
(43) Pub. Date: Oct. 26, 2006

(54) Methods and compositions for the treatment and prevention of degenerative joint disorders

(76) Inventors: Edward Fey, Boston, MA (US); Gregory D. Jay, Norfolk, MA (US)

Correspondence Address: CLARK & ELBING LLP, 101 FEDERAL STREET, BOSTON, MA, 02110, US

(21) Appl. No.: 10/559,551

(22) PCT Filed: Jun. 4, 2004
(86) PCT No.: PCT/US04/17946

§ 371 Date: Apr. 27, 2006

Related U.S. Application Data
(60) Provisional application No. 60/476,009, filed on Jun. 4, 2003.

(51) Int. Cl. A61K 36/06; A61K 36/886; A61K 36/899; A61K 36/28; A61K 36/10; A61K 36/20

(52) U.S. Cl. 424/195.15; 514/2; 424/750; 424/764; 424/771; 424/762; 424/773; 424/776; 424/744

(57) ABSTRACT

The present invention features methods and compositions for the treatment, reduction, and prevention of degenerative joint disorders by administering to a mammal a joint enhancing composition. According to this invention, the composition increases the level of expression and secretion of endogenous lubricin such that the joints in the mammal being treated are lubricated. This composition can be administered alone or in combination with one or more therapeutic agents.

### FIELD OF THE INVENTION

The present invention relates to methods and compositions for the treatment, reduction, and prevention of degenerative joint disorders, including osteoarthritis and rheumatoid arthritis.

### BACKGROUND OF THE INVENTION

In normal operations, joints (the sites where bones come together) function to provide smooth, painless, and stable force transmission between adjacent bones. The deterioration of joint function often results in chronic pain, lack of mobility and, in extreme cases, total disability and even death. In addition to physical injuries and age, a number of disorders can also affect the integrity of joints. Some disorders arise secondary to microbial infections while others are the result of autoimmune responses.

The most prevalent degenerative joint disorders are rheumatoid arthritis and osteoarthritis. Osteoarthritis is characterized by degenerative changes in the surface of the articular cartilage. Factors, which contribute to the development of osteoarthritis include, for example, family history, prior damage to the joint through injury or surgery, and the age of the individual (i.e., “wear and tear” damage of the articulating surfaces of the joint). Although osteoarthritis is particularly common in older age groups, this condition can also affect children. Rheumatoid arthritis, which is thought to be an autoimmune disease, is the result of an inflammation of the synovial membrane. In extreme cases, chronic inflammation erodes and distorts the joint surfaces and connective tissue resulting in severe articular deformity and chronic pain. Rheumatoid arthritis often leads to osteoarthritis, further compounding the destruction of the joint.

In both osteoarthritis and rheumatoid arthritis, the degeneration of the weight bearing joints, such as the hips and knees, can be especially debilitating and often requires surgery to relieve pain and increase mobility.

No means currently exist for halting or reversing the degenerative changes brought about by any of these disorders. Current treatment is instead directed at relieving pain and other symptoms associated with joint degeneration by administering to the patient analgesics and anti-inflammatory agents, for example. While such therapeutic agents are effective in alleviating arthritic symptoms, they often lead to severe side effects, including nausea and gastrointestinal ulceration. Thus, in spite of the chronic nature of most of these degenerative joint disorders, the long-term use of these drugs is often not recommended.

Thus, better treatment strategies are needed.

### SUMMARY OF THE INVENTION

The present invention is useful to treat, reduce, or prevent degenerative joint disorders (e.g., osteoarthritis, rheumatoid arthritis, synovitis, traumatic effusion, blunt trauma, juvenile arthritis, lupus, scleroderma, chondromalacia patellae, infectious arthritis, bursitis, tendinitis, fibrositis fibromyositis, polymyositis, canine arthritis, canine hip dysplasia, or equine degenerative joint disease) by administering to a mammal in need thereof (e.g., a human, dog, horse, or cat) a therapeutically effective amount of a joint enhancing composition adapted for oral administration. According to this invention, the administration of this composition increases the expression and secretion of endogenous lubricin by at least 10%, 20%, 30%, 40%, 50%, 60%, 70%, 80%, 90%, 100%, 200%, 300%, 500%, or more than 500% relative to an untreated control such that lubrication of the joints in the mammal is increased. By increasing the lubrication of joints, the degenerative joint disorder is in turn treated, reduced, or prevented as measured by any method known in the art. Typically, lubricin expression and secretion is increased in synovial fibroblasts but such expression and secretion may also be increased in any synovial cell of the mammal. Although degeneration of any joint is amenable to treatment with this composition, this invention is particularly useful to lubricate articulating joints such as a knee, hip, ankle, shoulder, or elbow.

The joint enhancing composition of the invention preferably contains at least two, three, five, seven, nine, eleven, thirteen, preferably fifteen, or more preferably all of the following elements: octacosanol (defatted wheat germ oil), elecampagne root (Linula), quercetin, L-cysteine, vitamin B1 (thiamin HCl), white oak bark (Quercus Alba), vitamin B5 (pantothenic acid, calcium D-pantothenate), aloe vera gel, black cohosh (Cimicifuga Racewosh), androstenedione, oat straw (Avena Sativa), oat straw (Avena Sativa) powder, L-Methionine, Shitake mushroom (Lentius Elodes), bromelain, horsetail (Equisetum), and borage oil (Borago Officianalis). For example, the composition may contain oat straw (Avena Sativa) (preferably between 15 and 25 mg, more preferably 21.5 mg), oat straw (Avena Sativa) powder (preferably between 150 and 170 mg, more preferably 160.0 mg), bromelain (2400 GDU) (preferably between 90 and 110 mg, more preferably 100.0 mg), pantothenic acid (Vitamin B5) (preferably between 30 and 40 mg, more preferably 35.0 mg), L-methionine (preferably between 25 to 40 mg, more preferably 33.0 mg), quercetin (preferably between 60 and 75 mg, more preferably 66.0 mg), horsetail silicic acid (preferably between 25.0 and 40.0 mg, more preferably 33.0 mg), and borage oil powder (preferably between 25 and 40 mg, more preferably 33.0 mg). Desirably, the oat straw SE is in an initial ratio of 10:1 and the initial concentration of the horsetail SE silicic acid is 1.5-3.0%. The borage oil powder is preferably admixed with gamma lipoic acid (GLA), more preferably at an initial concentration of 6.6%.

If desired, the composition of the invention may be co-formulated or administered with a second therapeutic agent, including for example, analgesics, antibiotics, antivirals, anti-inflammatories (e.g., non-steroidal anti-inflammatory drugs or corticosteroids), anti-neoplastics, anesthetics, enzymes, and immunosuppressive agents. Anti-inflammatory agents include, for example, ketoprofen, auranofin, naproxen, acetaminophen, aspirin, ibuprofen, phenylbutazone, indomethacin, sulindac, diclofenac, paracetamol, diflunisal, Celecoxib, and Rofecoxib. Exemplary corticosteroids are triamcinolone, hydrocortisone, fluticasone, and beclomethasone. Antimicrobial agents that can be formulated with the joint enhancing composition of the invention include antibiotics (e.g., clindamycin, minocycline, erythromycin, probenecid, or moxifloxacin), anti-fungal agents (e.g., nystatin or Amphotericin B), or anti-viral agents (e.g., acyclovir). Exemplary analgesics include procaine, lidocaine, tetracaine, dibucaine, benzocaine, p-buthylaminobenzoic acid 2-(diethylamino) ethyl ester HCl, mepivacaine, piperocaine, dyclonine, morphine, codeine, hydrocodone, and oxycodone. Other second therapeutic agents that can be co-formulated or administered with the composition are hyaluronic acid, methotrexate, gold (Myocrisin), sulphasalazine, chloroquine, glucosamine, or chondroitin. The second therapeutic agent may be administered within (either before or after) 14 days, 7 days, 24 hours, 12 hours, 1 hour, or simultaneously with the joint enhancing composition.

As used herein, by “lubricate the joint” is meant to smoothen the surfaces of the joints to provide smooth, painless, stable, force transmission between the bones that are connected by the joints.

By “arthritis” is meant a condition characterized by damage or inflammation in one or more joints. This condition is often manifested by pain, swelling, heat, redness, and the limitation of movements. The most common type of arthritis is osteoarthritis or ‘wear and tear arthritis.’ The condition results from the erosion of cartilage. Since the cartilage cannot be properly replaced, new bone structures will compensate for the loss of cartilage. This in turn produces bony swellings, which are painful because the new bone is stretching the sensitive lining of the pre-existing bone.

By “degenerative joint disorder” is meant any disorder, which is characterized by the degeneration of articular joints. Such disorders include, for example, the various forms of arthritis.

By “composition” is meant any mixture, which contains at least one therapeutically or biologically active agent and is suitable for administration to a patient. Any of the formulations of the invention can be prepared by well-known and accepted methods of art. See, for example, Remington: The Science and Practice of Pharmacy, 19^{th} edition, (ed. A R Gennaro), Mack Publishing Co., Easton, Pa., 1995.

By “therapeutically effective amount” is meant an amount sufficient to provide medical benefit. When administering a composition of the invention to a mammal according to the methods described herein, a therapeutically effective amount is usually about 0.1-4000 mg of the composition per dose. Preferably, the patient receives 0.5 mg, 1 mg, 10 mg, 50 mg, 100 mg, 250 mg, 500 mg, 750 mg, 1000 mg, 1500 mg, 2000 mg, 2500 mg, 3000 mg, 3500 mg, or 4000 mg of the composition in each dose. Dosing is typically performed 1-10 times each day.

By “treating” is meant administering a pharmaceutical composition for prophylactic and/or therapeutic purposes. The active ingredients of the pharmaceutical composition can treat the primary indication (e.g., joint degeneration) or secondary symptoms (e.g., concomitant pain or inflammation).

By “analgesic” is meant an agent, which relieves pain by elevating the pain threshold without significantly disturbing the consciousness of the patient.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

### DESCRIPTION OF THE FIGURES

FIG. 1 is a schematic diagram representing the exon arrangement of the MSF gene (GenBank U70136). Also shown are the binding sites for the anti-lubricin antibody and peanut agglutinin that were used in the lubricin detection assays.

FIG. 2 is a bar graph showing lubricin expression by synovial fibroblasts following the addition of various components of the nutritional supplement library.

### DETAILED DESCRIPTION

Degenerative joint disorders are progressive disorders of synovial joints characterized by articular cartilage degeneration and joint effusion. Acute or chronic trauma, overuse, developmental disease, joint instability, and old age can all lead to synovitis, impaired chondrocyte metabolism, and the formation of fissures in the joint cartilage. The synovial fluid of an inflamed or injured joint contains proteolytic enzymes such as trypsin, elastase, stromelysin, and hyaluronidase that are released into the joint where they degrade lubricating proteins or polypeptides in the synovial fluid and cartilage components resulting in pain and cartilage erosion. For example, infiltrating immune cells such as neutrophils secrete trypsin and/or elastase. Even a minor injury to an articulating joint or an inflammatory state can result in cellular infiltration and proteolytic enzyme secretion resulting in traumatic synovitis. Synovitis, in a period of a few days or weeks, can result in the loss of the cytoprotective layer of a joint, which in turn leads to loss of cartilage. Non-lubricated cartilaginous bearings may experience premature wear, which may initiate osteoarthritis. Individuals who clinically present with traumatic effusion (e.g., “water on the knee”) are predisposed to developing osteoarthritis since the elaboration of proteolytic enzymes degrades and depletes naturally-occurring lubricating compositions in the synovial fluid. Depletion of natural lubricating compositions occurs in other inflammatory joint disease such as rheumatoid arthritis.

Lubricating glycoprotein-1 (LGP-1), which was renamed lubricin, was initially isolated from bovine synovial fluid as a mucinous glycoprotein having the ability to lubricate articular cartilage in a manner equivalent to that of whole synovial fluid (Swann et al., (1981) Journal of Biological Chemistry 256 (11): 5921-5). Lubricin is a fragment formed as a result of the alternative splicing of exons 6 through 9 of the megakaryocyte-stimulating factor (MSF) gene and is secreted by synovial fibroblasts. Lubricin is homologous to the superficial zone protein (SZP), the N-terminal fragment of which is MSF (Jay et al., (2001) Journal of Orthopaedic Research 9(4):677-87). A lubricating glycoprotein (PSLF) with an apparent weight of 280 kDa purified from bovine synovial fluid is similar if not identical to lubricin (Jay et al., (2001) Journal of Orthopaedic Research 9(4): 677-87).

Here, we have identified a number of substances that upon oral administration to a mammal, results in the expression and secretion of lubricin, which replaces or supplements the synovial fluid of joints. Thus, the present invention is based on the discovery that administration of a therapeutically effective amount of a joint enhancing composition to a mammal increases the endogenous expression of lubricin such that the lubrication in the joints is increased. As a result of such administration, joint stiffness and pain are decreased and joint mobility is increased thereby treating, reducing, or preventing degenerative joint disorders. According to this invention, any articulating joint may be lubricated, including a knee, elbow, shoulder, hip, or any other weight-baring joint. Disorders amenable to the treatment according to the present invention include, for example, osteoarthritis, rheumatoid arthritis, synovitis, traumatic effusion, blunt trauma, juvenile arthritis, lupus, scleroderma, chondromalacia patellae, infectious arthritis, bursitis, tendinitis, fibrositis fibromyositis, and polymyositis, all of which are characterized by inflammation and pain in the joints, muscles, and related connective tissues. The methods of the present invention can also be used prophylactically to prevent future damage or degeneration of the joints. For example, a composition of the invention may be administered to athletes intermittently to minimize the risk of stress-related injury or cartilage degeneration.

Although the mammal being treated is preferably a human, other mammals amenable to treatment according to the present invention include dogs, horses, and cats. Canine osteoarthritis or canine hip dysplasia, for example, are prevalent clinical disorders that may be treated according to the present methods. Osteoarthritis afflicts an estimated one in five adult dogs, and an estimated 8 million dogs suffer from this degenerative potentially debilitating disease. While any dog can suffer from such a disorder, those most at risk are large breeds, geriatric dogs, very active dogs (such as working or sporting animals) and those with inherited joint abnormalities, such as hip or elbow dysplasia. The joint enhancing composition of the invention may therefore be formulated as a biscuit to facilitate administration to such mammals, and is described, for example in U.S. Pat. No. 6,524,609, hereby incorporated by reference. Equine degenerative joint disorders, such as osteoarthritis, are often a cause of lameness and impaired performance in horses and are also amenable to treatment according to the present invention.

Diagnosis of any of the degenerative joint disorders may be performed using any method known in the art, and is described in detail, for example in U.S. Ser. No. 09/298,970, hereby incorporated by reference. Disorders may be diagnosed, for example, by physical examination, by the detection of inflammation in the synovial joints, or by the detection of molecular markers characteristic of such disorders in a biological sample collected from the mammal, such as synovial fluid, blood, serum, or urine.

To assess whether the present invention is useful to treat, reduce, or prevent a degenerative joint disorder, any method known in the art may be used. For example, a medically desirable result may be a reduction of pain (measured, e.g., using a visual analog pain scale, described for example, by Peyron et al. (1993) J. Rheumatol 20 (suppl. 39): 10-15) or increased joint mobility (measured, e.g., using pedometry as described in Belcher et al. (1997) J Orthop. Trauma 11:106-109).

Another method to measure lubricity of synovial fluid following treatment is to aspirate a small volume of synovial fluid from the affected joint and test the lubricating properties of the fluid in vitro using a friction apparatus. All these methods to test the efficacy of treatments are described in U.S. Ser. No. 09/298,970.

Therapeutic Agents

According to this invention, the administration of a joint enhancing composition increases the expression of endogenous expression of lubricin in the synovial joints such that the lubrication of the joints is increased. Desirably, the expression of endogenous lubricin is increased by at least 10%, 20%, 30%, 40%, 50%, 60%, 70%, 80%, 90%, 100%, 200%, 300%, 500%, or more than 500% compared to an untreated control. The composition preferably contains two, three, five, seven, nine, eleven, thirteen, more preferably fifteen, or more preferably all of the substances of table 1. For example, this composition may contain the following components: oat straw (Avena Sativa) (between 15.0 and 25.0 mg, preferably 21.5 mg), oat straw (Avena Sativa) powder (between 150.0 and 170.0 mg, preferably 160.0 mg), Bromelain (2400 GDU) (between 90.0 and 110.0 mg, preferably 100.0 mg), Pantothenic acid (Vitamin B5) (between 30.0 and 40.0 mg, preferably 35.0 mg), L-methionine (between 25.0 and 40.0 mg, preferably 33.0 mg), Quercetin (between 60.0 and 75.0 mg, preferably 66.0 mg), horsetail SE silicic acid (between 25.0 and 40.0 mg, preferably 33.0 mg), and borage oil powder (between 25.0 and 40.0 mg, preferably 33.0 mg). Desirably, the oat straw SE is in an initial ratio of 10:1 and the initial concentration of the horsetail SE silicic acid is 1.5-3.0%. The borage oil powder is preferably admixed with GLA, more preferably at an initial concentration of 6.6%. The components are present in the compositions of the invention in varying amounts depending on the nature and condition of the joint degenerative condition being treated, the anticipated frequency and duration of therapy, and the type of pharmaceutical composition used to deliver joint enhancing composition. Typically, therapy is designed to deliver between 0.1 and 4000 mg of the composition per day to the patient. Preferably, the patient receives 0.5 mg, 1 mg, 10 mg, 50 mg, 100 mg, 250 mg, 500 mg, 750 mg, 1000 mg, 1500 mg, 2000 mg, 2500 mg, 3000 mg, 3500 mg, or 4000 mg of the composition between one to ten times per day.

Although the administration of the joint enhancing composition of the invention lubricates synovial joints by increasing endogenous lubricin expression, the components of the present composition may also act synergistically or collectively to provide any of the following therapeutic benefits: to provide sufficient sources of necessary metabolic precursors for the repair and maintenance of connective tissues, to ensure the proper absorption of these metabolic precursors in the digestive tract, to diminish the inflammatory response in the affected area so that the connective tissue degradation process is halted and repair is initiated; to suppress the autoimmune response and any further degradation of tissue in the affected area; and to stimulate the blood circulatory system, which simultaneously enhances the delivery of the metabolic precursors to the affected areas and removes deleterious deposits in the affected areas.

Secondary Therapeutic Agents

In addition to a joint enhancing composition, the mammal being treated according to the present invention may also be administered with a secondary therapeutic agent. The second therapeutic agent may be administered within 3 days, 1 day, 12 hours, 1 hour, or simultaneously with the composition. The second therapeutic agent may or may not be administered using the same route of administration as the composition. Alternatively, the second therapeutic agent can be present in the same pharmaceutical composition as the joint enhancing composition. Exemplary secondary therapeutic agents are provided below.

Anti-Inflammatory Agents

Any suitable anti-inflammatory agent (e.g., non-steroidal anti-inflammatory drugs, NSAIDs) may be co-formulated with the joint enhancing composition or administered to the mammal being treated with this composition at concentrations known to be effective for these agents. Many of the most useful anti-inflammatory agents also have analgesic and/or antipyretic properties. NSAIDs, for example, are used to reduce the formation of the prostaglandins responsible for the pain and inflammation associated with arthritis. They can reduce pain swelling and joint stiffness, as well as improve mobility. Anti-inflammatory agents suitable for co-formulation with the composition of the invention include, for example, ketoprofen, auranofin, naproxen, acetaminophen, aspirin (acetylsalicylic acid), ibuprofen, phenylbutazone, indomethacin, sulindac, diclofenac, paracetamol, and diflunisal, Celecoxib, and Rofecoxib.

Steroids

Steroids are commonly used to treat degenerative joint disorders, such as arthritis, to suppress the immune system and symptoms of inflammation. Typically, such agents are used in severe cases of osteoarthritis and are usually administered orally or by injection. Occasionally, steroids may also be injected directly into an affected joint. Exemplary steroids include, for example, triamcinolone, hydrocortisone, fluticasone, or beclomethasone.

Analgesics and Anesthetics

Any of the commonly used analgesics can be used in the compositions of the invention. Examples of useful anesthetics include procaine, lidocaine, tetracaine, dibucaine, benzocaine, p-buthylaminobenzoic acid 2-(diethylamino) ethyl ester HCl, mepivacaine, piperocaine, dyclonine, and opioids (e.g., morphine, codeine, hydrocodone, and oxycodone).

Antimicrobial Agents

Because degenerative joint diseases may also occur concomitantly with microbial infections, the joint enhancing composition of the invention may also be administered with an antimicrobial agent. Infectious arthritis, for example, is characterized by joint pain, soreness, stiffness and swelling caused by an infection by any of several types of agents, including bacteria, viruses and even fungi. These infections can affect a joint after spreading through the bloodstream from another part of the body, such as the lungs during pneumonia. An infection also can enter a joint through a nearby wound. Sometimes, the tissue surrounding the joint becomes infected after surgery, an injection or trauma (e.g., an insect bite). Once the infectious agent reaches the joint, it can cause symptoms of joint inflammation and, at times, fever and chills. Depending on the type of infection, one or more joints may be affected. Accordingly, the mammal being treated with the composition of the invention may also be administered with an antibiotic, an anti-viral agent, or an anti-fungal agent.

Exemplary antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin. Particularly useful antibiotics include clindamycin, minocycline, erythromycin, probenecid, and moxifloxacin.

Antiviral agents are substances capable of destroying or suppressing the replication of viruses. Examples of anti-viral agents include 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, benzoic acid, undecylenic alkanolamide, ciclopirox olamine, polyenes, imidazoles, allylamine, thicarbamates, amphotericin B, butylparaben, clindamycin, econaxole, fluconazole, flucytosine, griseofulvin, nystatin, and ketoconazole.

Other Second Therapeutic Agents

Any therapeutic agent that is typically used in the treatment, prevention, and reduction of degenerative joint disorders may also be administered or co-formulated with the composition of the invention.

Exemplary agents include oral glucosamine and chondroitin, which are often used as they form the building blocks of cartilage, the substance that lines the joints; hyaluronic acid; methotrexate; disease modifying drugs (e.g., Gold (Myocrisin), Sulphasalazine, and Chloroquine); and natural remedies (e.g., sea cucumber extract, shark cartilage, green lipped mussel, evening primrose oil, and cod liver oil).

Pharmaceutical Formulations

Joint enhancing compositions suitable for ingestion include, for example, a pill, capsule, tablet, emulsion, solution, suspension, syrup, or soft gelatin capsule. Additionally, the pharmaceutical formulations may be designed to provide either immediate or controlled release of the antibiotic upon reaching the target site. The selection of immediate or controlled release compositions depends upon a variety of factors including the severity of the joint degeneration. Methods well known in the art for making formulations are found, for example, in Remington: The Science and Practice of Pharmacy (20th ed.), ed. A. R. Gennaro, 2000, Lippincott Williams & Wilkins, Philidelphia, or in Encyclopedia of Pharmaceutical Technology, eds. J. Swarbrick and J. C. Boylan, 1988-1999, Marcel Dekker, New York.

Dosages

All of the therapeutic agents employed in the oral compositions of the present invention, including each substance of the joint enhancing composition, can be used in the dose ranges currently known and used for these agents. The following are illustrative examples of dose ranges for the active ingredients of the compositions of the invention. Different concentrations of either each substance of the joint enhancing composition or the other agents may be employed depending on the clinical condition of the patient, the site of joint degeneration, and the severity of the damage. Additional considerations in dose selection include: disease etiology, patient age (pediatric, adult, geriatric), general health and comorbidity.

Identification of Lubricin-Stimulating Substances

To identify substances having the ability to lubricate synovial joints, we first screened a library of substances for the ability to induce expression and secretion of endogenous lubricin in cultured synovial fibroblasts. A library of nutritional supplements containing 175 natural compounds was made using a natural compound collection. Each supplement was dissolved in water to an approximate concentration of 10 mM. 96-well daughter plates were made and plates were stored at −20° C.

Human synovial fibroblasts were plated the evening of the first day of the assay and grown overnight. In the morning of day 2, a supplement solution was added to each well of the 96-well plate. Each supplement was tested in 6 different wells to provide enough separate measurements to allow a statistical analysis of the results of the assay. After 48-72 hours, cells were lysed using a non-ionic detergent and supernatants containing cellular extracts were collected. Each supernatant was assayed for lubricin expression using any of the two methods described below.

We have developed two methods to measure the expression of lubricin, both of which are based on a sandwich assay. In the first sandwich assay, concanavilin A (a lectin) is used to capture lubricin while peanut agglutinin (a lectin) is used to detect proteins secreted by synovial fibroblasts. In contrast, the second assay relies on a rabbit polyclonal antibody for both the capture and detection of lubricin secreted by fibroblasts. In both assays, purified lubricin was used as a calibrator.

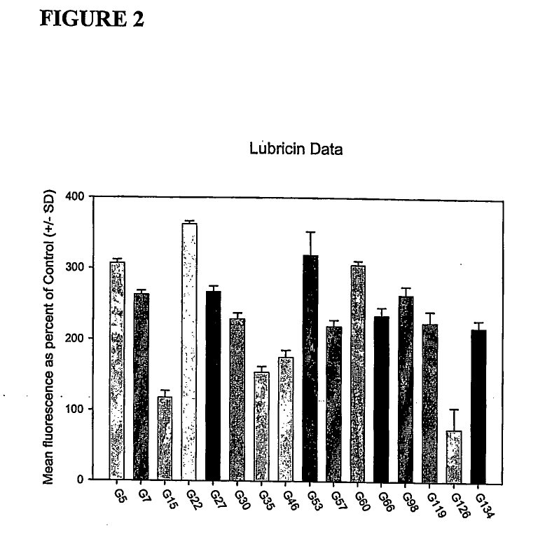

A substance that raised the level of measured lubricin in a significant manner when analyzed by t-test was considered a potentially efficacious substance useful for the present invention. All significant results were repeated in two additional experiments. Of particular importance to this discovery is the observation that of the 175 nutritional supplements tested in each lubricin detection assay, only 16 supplements were observed to increase lubricin expression in a significant manner. In this regard, the 16 compounds that increased lubricin in the lectin-based assay were the same 16 compounds that increased lubricin levels in the antibody-based assay. Taken together, our results indicate that the lubricin measured in this assay is a glycosylated form of the protein. Compounds that increase lubricin expression in synovial fibroblasts are indicated in Table 1 and the level of increase in lubricin expression is shown in Table 2 and FIG. 2.

TABLE 1
Summary of evaluation of hits in antibody based assay

| BRD No. | Supplement Name | Alternate Name | Supplier |
|---|---|---|---|
| G5 | Octacosanol | Defatted Wheat Germ Oil | Solgar |
| G7 | Elecampane Root | Linula | Nature's |
| | | | Way |
| G15 | Quercetin | Quercetin | Twin Lab |
| G22 | L-Cysteine | L-Cysteine | Twin Lab |
| G27 | Vitamin B1 | Thiamin HCl | GNC |
| G30 | White Oak Bark | Quercus Alba | Solaray |
| G-35 | Pantothenic Acid | Calcium d-Pantothenate | GNC |
| G-46 | Aloe Vera Gel | Aloe Vera Gel | GNC |
| G-53 | Black Cohosh | Cimicifuga Racewosh | GNC |
| G-57 | Androstenedione | Androstenedione | GNC |
| G-60 | Avena Sativa | Oat Straw | GNC |
| G-66 | L-Methionine | L-Methionine | Solgar |
| G-98 | Shitake Mushroom | Lentius Elodes | GNC |
| G-119 | Horse Tail | Equisetum | GNC |
| G-126 | Bromelain | Bromelain | GNC |
| G-134 | Borage Oil | Borago Officianalis | CNC |

TABLE 2
Lubricin Expression

| | Percent stimulation relative to | |
|---|---|---|
| | untreated control Adjusted for | |
| Compound | background | Mann Whitney Rank Sum |
| G-5 | 307% | P < 0.001 |
| G-7 | 263% | P < 0.001 |
| G-15 | 118% | P = 0.606 |
| G-22 | 363% | P < 0.001 |
| G-27 | 267% | P < 0.001 |
| G-30 | 229% | P < 0.001 |
| G-35 | 154% | P < 0.001 |
| G-46 | 176% | P < 0.001 |
| G-53 | 319% | P < 0.001 |
| G-57 | 219% | P < 0.001 |
| G60 | 306% | P < 0.001 |
| G66 | 234% | P < 0.001 |
| G98 | 263% | P < 0.001 |
| G119 | 224% | P < 0.001 |
| G126 | 75% | P = 0.076 |
| G134 | 218% | P < 0.001 |

Lubricin Detecting Assays

Substances from the nutritional supplement library were screened for their ability to stimulate lubricin expression in primary human synovial fibroblasts. Cells were cultured in the presence or absence of each substance and lubricin expression was measured using the two detection assays discussed below.

In both assays, the cellular content of synovial cells was extracted as follows. Following the removal of culture media, cells were washed twice with D-PBS. Cells were first incubated in 500 μL of 1% Triton in D-PBS for five minutes and then mechanically disrupted using a pipette to ensure that all cells detach from the plates. The Triton solution containing the cells was transferred to an eppendorf and stored on ice. Samples were sonicated for three seconds and centrifuged for five minutes. The supernatant containing the cellular extracts was transferred to a fresh tube and tested for lubricin expression.

Lectin-Based Assay

96-well plates were coated with 100 μg/mL of concavallin A in phosphate buffer saline (PBS) for one hour at room temperature and dried overnight. The next day, plates were washed twice with PBS.

200 μL of the cellular extract-containing supernatant was added in each well. 100 μL of supernatant obtained from the cell cultures (before cell lysis), 100 μL of fresh media, and 100 μL of media (DMEM) without fetal bovine serum were also added to individual wells in the plate. In order to generate a protein standard curve, various known amounts of purified lubricin were also assayed. In this assay, each sample was placed in six different wells for statistical significance.

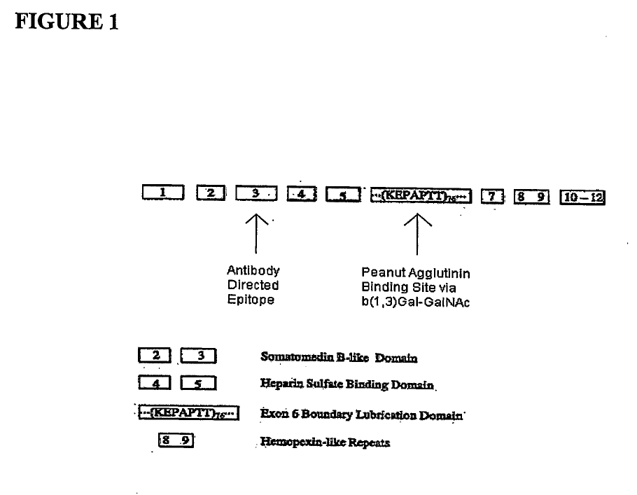

Plates were incubated for one hour at room temperature after which the contents of each well was emptied. Wells were washed twice with PBS and 0.5 μg/mL of peanut agglutinin (Arachis hypogea) conjugated to FITC was added to each well. The binding site of peanut agglutinin in the lubricin protein is shown in FIG. 1.

Plates were incubated for one hour at room temperature with shaking after which wells were washed twice with PBS. The level of lubricin expression was determined by fluorescent spectrophotometer at an absorbance of 535 nm.

Antibody-Based Detection Assay

96-well plates were coated with 100 μL of an anti-lubricin antibody solution (1:10,000 dilution in carbonate buffer pH 9.0) for one hour at room temperature. The antibody solution was removed and replaced with 200 μL/well of 3% bovine serum albumin (BSA) in carbonate buffer pH 9.0 for one hour at room temperature. The wells were washed twice with 250 μL of PBS (+0.05% Tween-20). The cellular extract-containing supernatants were diluted in 1% BSA containing PBS and added to each well as described above. Plates were incubated for one hour at room temperature, after which wells were washed twice with 250 μL of PBS (+0.05% Tween-20). 100 μL of 50 μg/mL of Arachis Hypogea-FITC (diluted in 1% BSA containing PBS) was added to each well and plates were incubated for one hour at room temperature. Wells were washed twice with 250 μL of PBS (+0.05% Tween-20), and washed again twice with 100 μL of PBS. Using a standard curve, the level of lubricin expression was determined by fluorescent spectrophotometer at an absorbance of 535 nm.

The following examples are intended to illustrate the principle of the present invention and circumstances when the joint enhancing composition is indicated. The following examples are not intended to be limiting.

### EXAMPLE 1

### Treatment of a Patient Suffering from Rheumatoid Arthritis in an Articular Region

A patient suffering from rheumatoid arthritis in the hips is treated twice a day, every day, with 200.0 mg of a joint enhancing composition (containing 25.0 mg oat straw (Avena Sativa) SE 10:1, 155.0 mg oat straw (Avena Sativa) powder, 100.0 mg Bromelain (2400 GDU), 35.0 mg Pantothenic acid (Vitamin B5), 33.0 mg L-methionine, 60.0 mg Quercetin, 26.0 mg horsetail SE 1.5-3.0% silicic acid, and 25.0 mg borage oil powder (6.6% GLA-Bioriginal)). If desired, the patient may also take ibuprofen to reduce pain to the joints.

### EXAMPLE 2

### Treatment of Osteoarthritis

A geriatric patient diagnosed with osteoarthritis is administered twice a day, everyday, with 100.0 mg of a joint enhancing composition (containing 30.0 mg oat straw (Avena Sativa) SE 10:1, 155.0 mg oat straw (Avena Sativa) powder, 110.0 mg Bromelain (2400 GDU), 35.0 mg Pantothenic acid (Vitamin B5), 33.0 mg L-methionine, 60.0 mg Quercetin, 26.0 mg horsetail SE 1.5-3.0% silicic acid, and 25.0 mg borage oil powder (6.6% GLA-Bioriginal)). Because the patient suffers from pain mainly in the right knee, cortisone is also injected into this knee to alleviate the pain.

### EXAMPLE 3

### Treatment of Infectious Arthritis

Staphylococcus bacteria are common bacteria that can cause infections through cuts or other breaks in the skin, or through contaminated food. The bacteria can be released in the bloodstream and spread to the knee or other joints, causing intense and sudden pain, swelling and immobility of the joint. Because joint damage can develop in a matter of days if the infection is not promptly detected and treated, patients contaminated with such a bacteria are immediately administered daily with a joint enhancing composition using one of the above formulations in addition to treatment with an antibiotic. Treatment is continued for 2 weeks.

### EXAMPLE 4

### Treatment of Osteoarthritis

An athlete is diagnosed with osteoarthritis of the right foot with severe pain on running. The patient is administered with 300.0 mg of the composition of the invention twice daily according to any of the above formulations and 50 mg of chondroitin sulfate, which is taken three times a day. Treatment is continued for four months until the pain subsides. A maintenance dose of 50.0 mg of a joint enhancing composition is continued for five months after the pain has subsided.

What is claimed is:

1. A joint enhancing composition adapted for oral administration, wherein said composition increases the endogenous expression of lubricin by at least 10% relative to an untreated control.

2. The joint enhancing composition of claim 1, wherein said composition comprises at least two substances selected from the group consisting of octacosanol (defatted wheat germ oil), elecampagne root (Linula), quercetin, L-cysteine, vitamin B 1 (thiamin HCl), white oak bark (Quercus Alba), vitamin B5 (pantothenic acid, calcium D-pantothenate), aloe vera gel, black cohosh (Cimicifuga Racewosh), androstenedione, oat straw (Avena Sativa), oat straw (Avena Sativa) powder, L-Methionine, Shitake mushroom (Lentius Elodes), bromelain, horsetail (Equisetum), and borage oil (Borago Officianalis).

3. The joint enhancing composition of claim 2, wherein said composition contains at least three of said substances.

4. The joint enhancing composition of claim 3, wherein said composition contains at least five of said substances.

5. The joint enhancing composition of claim 4, wherein said composition contains at least seven of said substances.

6. The joint enhancing composition of claim 5, wherein said composition contains at least nine of said substances.

7. The joint enhancing composition of claim 6, wherein said composition contains at least eleven of said substances.

8. The joint enhancing composition of claim 7, wherein said composition contains at least thirteen of said substances.

9. The joint enhancing composition of claim 8, wherein said composition contains at least fifteen of said substances.

10. The joint enhancing composition of claim 9, wherein said composition contains octacosanol (defatted wheat germ oil), elecampagne root (Linula), quercetin, L-cysteine, vitamin B 1 (thiamin HCl), white oak bark (Quercus Alba), vitamin B5 (pantothenic acid, calcium D-pantothenate), aloe vera gel, black cohosh (Cimicifuga Racewosh), androstenedione, oat straw (Avena Sativa), oat straw (Avena Sativa) powder, L-Methionine, Shitake mushroom (Lentius Elodes), bromelain, horsetail (Equisetum), and borage oil (Borago Officianalis).

11. The joint enhancing composition of claim 1, comprising oat straw (Avena Sativa) SE, oat straw (Avena Sativa) powder, bromelain, vitamin B5 (pantothenic acid, calcium D-pantothenate), L-methionine, quercetin, horsetail (Equisetum), and borage oil (Borago Officianalis).

12. The joint enhancing composition of claim 11, comprising:
from 15 to 25 mg of oat straw (Avena Sativa),
from 150 to 170 mg of oat straw (Avena Sativa) powder,
from 90 to 110 mg of bromelain (2400 GDU),
from 30 to 40 mg of vitamin B5 (pantothenic acid, calcium D-pantothenate),
from 25 to 40 mg of L-methionine,
from 60 to 75 mg of quercetin,
from 25 to 40 mg of horsetail SE silicic acid, and
from 25 to 40 mg borage oil powder.

13. The joint enhancing composition of claim 12, comprising:
21.5 mg of oat straw (Avena Sativa),
160.0 mg of oat straw (Avena Sativa) powder,
100.0 mg of bromelain (2400 GDU),
35.0 mg of vitamin B5 (pantothenic acid, calcium D-pantothenate),
33.0 mg of L-methionine,
66.0 mg of quercetin,
33.0 mg of horsetail SE silicic acid, and
33.0 mg of borage oil powder.

14. The joint enhancing composition of claim 12, wherein said oat straw SE is in an initial 10:1 ratio.

15. The joint enhancing composition of claim 12, wherein the initial concentration of said horsetail SE silicic acid is 1.5-3.0%.

16. The joint enhancing composition of claim 12, wherein said borage oil powder is in gamma lipoic acid (GLA).

17. The joint enhancing composition of claim 12, wherein the initial concentration of said borage oil powder is 6.6%.

18. The joint enhancing composition of claim 1, further comprising a second therapeutic agent.

19. The joint enhancing composition of claim 18, wherein said second therapeutic agent is selected from the group consisting of analgesics, antibiotics, antivirals, anti-inflammatories, anesthetics, enzymes, and immunosuppressive agents.

20. The joint enhancing composition of claim 19, wherein said anti-inflammatory is a non-steroidal anti-inflammatory drug or a corticosteroid.

21. The joint enhancing composition of claim 20, wherein said corticosteroid is triamcinolone, hydrocortisone, fluticasone, or beclomethasone.

22. The joint enhancing composition of claim 19, wherein said anti-inflammatory agent is ketoprofen, auranofin, naproxen, acetaminophen, aspirin, ibuprofen, phenylbutazone, indomethacin, sulindac, diclofenac, paracetamol, diflunisal, Celecoxib, or Rofecoxib.

23. The joint enhancing composition of claim 19, wherein said antibiotic is clindamycin, minocycline, erythromycin, probenecid, or moxifloxacin.

24. The joint enhancing composition of claim 19, wherein said wherein said anti-fungal agent is nystatin or Amphotericin B.

25. The joint enhancing composition of claim 19, wherein said anti-viral agent is acyclovir.

26. The joint enhancing composition of claim 19, wherein said analgesic is procaine, lidocaine, tetracaine, dibucaine, benzocaine, p-buthylaminobenzoic acid 2-(diethylamino) ethyl ester HCl, mepivacaine, piperocaine, dyclonine, morphine, codeine, hydrocodone, or oxycodone.

27. The joint enhancing composition of claim 18, wherein said second therapeutic agent is hyaluronic acid, methotrexate, Gold (Myocrisin), Sulphasalazine, Chloroquine, glucosamine, or chondroitin.

28. A method of lubricating a joint in a mammal by administering to said mammal a therapeutically effective amount of a joint enhancing composition adapted for oral administration, wherein said composition increases the endogenous expression of lubricin by at least 10% relative to an untreated control.

29. The method of claim 28, wherein said joint is an articulating joint.

30. The method of claim 29, wherein said articular joint is a knee, hip, ankle, shoulder, or elbow.

31. The method of claim 28, wherein said mammal is a human, a dog, or a horse.

32. The method of claim 28, wherein said increase in endogenous expression of lubricin is in synovial cells of said joint.

33. The method of claim 32, wherein said cells are fibroblasts.

34. A method of treating, reducing, or preventing a degenerative joint disorder by administering to a mammal in need thereof a therapeutically effective amount of a joint enhancing composition adapted for oral administration, wherein said composition increases the endogenous expression of lubricin by at least 10% relative to an untreated control.

35. The method of claim 34, wherein said disorder is osteoarthritis, rheumatoid arthritis, juvenile arthritis, blunt trauma, synovitis, traumatic effusion, lupus, scleroderma, chondromalacia patellae, infectious arthritis, bursitis, tendinitis, fibrositis fibromyositis, or polymyositis.

36. The method of claim 35, wherein said increase in endogenous expression of lubricin is in synovial cells of said joint.

37. The method of claim 36, wherein said cells are fibroblasts.

38. The method of claim 34, wherein a second therapeutic agent is administered to said mammal.

39. The method of claim 38, wherein said second therapeutic agent is selected from the group consisting of analgesics, antibiotics, antivirals, anti-inflammatories, anesthetics, enzymes, and immunosuppressive agents.

40. The method of claim 39, wherein said anti-inflammatory is a non-steroidal anti-inflammatory drug or a corticosteroid.

41. The method of claim 40, wherein said corticosteroid is triamcinolone, hydrocortisone, fluticasone, or beclomethasone.

42. The method of claim 39, wherein said anti-inflammatory agent is ketoprofen, auranofin, naproxen, acetaminophen, aspirin, ibuprofen, phenylbutazone, indomethacin, sulindac, diclofenac, paracetamol, diflunisal, Celecoxib, or Rofecoxib.

43. The method of claim 39, wherein said antibiotic is clindamycin, minocycline, erythromycin, probenecid, or moxifloxacin.

44. The method of claim 39, wherein said wherein said anti-fungal agent is nystatin or Amphotericin B.

45. The method of claim 39, wherein said anti-viral agent is acyclovir.

46. The method of claim 39, wherein said analgesic is procaine, lidocaine, tetracaine, dibucaine, benzocaine, p-buthylaminobenzoic acid 2-(diethylamino) ethyl ester HCl, mepivacaine, piperocaine, dyclonine, morphine, codeine, hydrocodone, or oxycodone.

47. The method of claim 38, wherein said second therapeutic agent is hyaluronic acid, methotrexate, Gold (Myocrisin), Sulphasalazine, Chloroquine, glucosamine, or chondroitin.

48. The method of claim 38, wherein said composition and said second therapeutic are administered in the same formulation.

49. The method of claim 38, wherein said composition and said second therapeutic are administered in different formulations.

50. The method of claim 49, wherein said composition and said second therapeutic are administered within 14 days of each other.

51. The method of claim 50, wherein said composition and said second therapeutic are administered within 24 hours of each other.

52. The method of claim 34, wherein said mammal is a human.

53. The method of claim 34, wherein said mammal is a dog.

54. The method of claim 53, wherein said degenerative joint disorder is canine arthritis or canine hip dysplasia.

55. The method of claim 34, wherein said mammal is a horse.

56. The method of claim 55, wherein said degenerative joint disorder is equine degenerative joint disease.
